(19) United States
(12) Patent Application Publication
(10) Pub. No.: US 2010/0317681 A1
(43) Pub. Date: Dec. 16, 2010

(54) MODULATION OF SYSTEMIC EXPOSURE TO RIFAXIMIN

(75) Inventor: William Forbes, Raleigh, NC (US)

Correspondence Address: EDWARDS ANGELL PALMER & DODGE LLP, P.O. BOX 55874, BOSTON, MA, 02205, US

(73) Assignee: Salix Pharmaceuticals, Ltd., Morrisville, NC (US)

(21) Appl. No.: 12/508,864

(22) Filed: Jul. 24, 2009

Related U.S. Application Data
(60) Provisional application No. 61/187,251, filed on Jun. 15, 2009.

(51) Int. Cl. A61K 31/439

(52) U.S. Cl. 514/279

(57) ABSTRACT

The present invention relates to the effect of hepatic insufficiency on the pharmacokinetics of rifaximin. Also provided are methods of determining an appropriate dose of rifaximin for a subject suffering from hepatic insufficiency. In addition, methods of treatment are provided subjects having or susceptible to hepatic insufficiency to be treated with rifaximin.

### RELATED APPLICATIONS

This application claims the benefit of U.S. Provisional Application 61/187,251, filed Jun. 15, 2009, the entire contents of which are expressly incorporated herein by reference.

### BACKGROUND OF THE INVENTION

Rifaximin (INN; see The Merck Index, XIII Ed., 8304) is an antibiotic belonging to the rifamycin class of antibiotics, e.g., a pyrido-imidazo rifamycin. It has been reported that rifaximin is characterized by a negligible systemic absorption, due to its chemical and physical characteristics (Descombe J. J. et al. Pharmacokinetic study of rifaximin after oral administration in healthy volunteers. Int J Clin Pharmacol Res, 14 (2), 51-56, (1994)).

Rifaximin is described in Italian Patent IT 1154655 and EP 0161534, both of which are incorporated herein by reference in their entirety for all purposes. EP 0161534 discloses a process for rifaximin production using rifamycin O as the starting material (The Merck Index, XIII Ed., 8301).

### SUMMARY OF THE INVENTION

One embodiment is a method of treating Travelers' Diarrhea (TD) in a subject. The method includes: administering rifaximin to a subject suffering from Travelers' Diarrhea; and informing the subject that systemic plasma exposure to rifaximin is increased in subjects suffering from hepatic insufficiency in comparison to subjects not suffering from hepatic insufficiency.

Another embodiment is a method of using rifaximin for treating a patient's condition. The embodiment includes providing a patient with rifaximin and informing the patient or a medical care worker that systemic plasma exposure to rifaximin is increased in patients suffering from hepatic insufficiency, and that administration of rifaximin to a patient with hepatic insufficiency can affect plasma concentration, safety, or efficacy of rifaximin.

Yet another embodiment includes a method of treating a subject suffering from an indication treatable by rifaximin. This method includes administering rifaximin to the subject and advising the subject that systemic plasma exposure to rifaximin is increased in subjects suffering from hepatic insufficiency in comparison to subjects not suffering from hepatic insufficiency.

One other embodiment is a method that includes selecting a subject at risk for hepatic insufficiency, and treating the subject with rifaximin, wherein systemic plasma exposure to rifaximin is increased following the treatment in comparison to a subject without hepatic insufficiency.

Other embodiments and aspects are disclosed infra.

### BRIEF DESCRIPTION OF THE DRAWINGS

FIG. 1 is a graph showing the mean +SD of the plasma concentration-time profiles of rifaximin on a linear scale.

### DETAILED DESCRIPTION OF THE PREFERRED EMBODIMENT

Embodiments provided herein relate to the discovery of the difference in systemic exposure, plasma concentration, and terminal elimination rate constant of rifaximin in a subject suffering from hepatic insufficiency as compared to a subject having normal liver function. Further, the invention relates to the discovery of the effect of food on the time to reach maximum plasma concentrations of rifaximin in a subject.

Embodiments provided herein also relate to administration of medicinal preparations to a subject in need of treatment with compositions, such as rifaximin.

Rifaximin is a compound of the rifamycin class of antibiotics. Rifaximin is a compound having the structure of Formula I:

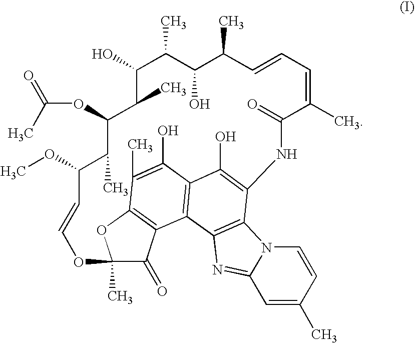

In one embodiment, rifaximin is a poorly absorbed molecule in certain subjects. This is shown in clinical pharmacokinetic studies in normal subjects following single and multiple oral doses demonstrate that rifaximin is poorly absorbed from the gastrointestinal tract (e.g., <1% of the drug is absorbed after oral administration). While only trace amounts of the parent drug and metabolites were detected in urine, there was high fecal recovery of rifaximin primarily as unchanged drug. Upon repeated administration, there was little or no systemic drug accumulation and the time to reach steady state was short. Unexpectedly, administration of a 1.5 fold higher daily dose in the TID regimen did not result in a commensurate increase in total daily systemic exposure to rifaximin; the AUC_{Total} was only approximately 13% higher after TID dosing than after BID dosing.

As used herein, the term “systemic exposure” is intended to mean the exposure of a subject to rifaximin by the administration and subsequent plasma exposure to rifaximin in the subject followed by the distribution throughout the subject's body. Increased systemic exposure can be measured by determining the plasma concentration of rifaximin in a subject. In exemplary embodiments, increased systemic exposure can be due to a decrease in the clearance of rifaximin.

A combination of physiopathological factors may explain the increase in rifaximin exposure in subjects with a history of HE as compared to healthy subjects. Hepatic encephalopathy episodes result from central nervous system accumulation of nitrogenous substances derived from the gut (primarily ammonia) due to porto-systemic shunts and portal hypoperfusion associated with hepatic cirrhosis. Subjects having mild and moderate impairment of liver function and a documented history of hepatic encephalopathy episodes were enrolled in the study. Hepatic encephalopathy is a complication of cirrhosis resulting from portal hypertension, cerebral vasodilatation, and hepatic insufficiency. Recent studies showed high prevalence of spontaneous portal-systemic shunts in persistent hepatic encephalopathy.

In chronic liver disease, a substantial part of the portal circulation does not perfuse functional liver cells due the presence of intrahepatic functional shunts or extrahepatic anatomic shunts. As a result, first pass clearance of orally administered agents is reduced and systemic bioavailability is increased. The higher exposure values of rifaximin were likely, in part due to a reduction of presystemic metabolism in the presence of porto-systemic shunts and/or portal hypoperfusion. This possibility is supported by the fact that although the plasma exposure to rifaximin from the gastrointestinal tract is limited (˜0.32% of the dose), it is a hypothesis that this small absorbed fraction undergoes first-pass metabolism in healthy subjects (˜90.6%), since only a small proportion of unchanged rifaximin is excreted in urine (0.03% of the dose). Additionally, since the rifaximin terminal t_{1/2} was clinically statistically significantly longer (about 2-fold) in HE subjects relative to healthy subjects, higher exposure found in HE may be due to a reduction in the systemic clearance of the drug.

As used herein, “subject” includes organisms which are capable of suffering from a bowel disorder or other disorder treatable by rifaximin or who could otherwise benefit from the administration of a rifaximin product as described herein, such as human and non-human animals. The term “non-human animals” provided herein includes all vertebrates, e.g., mammals, e.g., rodents, e.g., mice, and non-mammals, such as non-human primates, e.g., sheep, dog, cow, chickens, amphibians, reptiles, etc.

“Susceptible to a bowel disorder,” as used herein, includes subjects at risk of developing a bowel disorder, e.g., subjects suffering from HE, immune suppression, subjects that have been exposed to other subjects with a bacterial infection, subjects with a family history of a bowel disorder, subjects with a gene profile indicating disease or risk of bowel disorder, subjects with known susceptibility to bowel disorder, physicians, nurses, subjects traveling to remote areas known to harbor bacteria that causes travelers' diarrhea, etc.

A subject “suffering from hepatic insufficiency” as used herein includes subjects diagnosed with a clinical decrease in liver function, for example, due to hepatic encephalopathy, hepatitis, or cirrhosis. Hepatic insufficiency can be quantified using any of a number of scales including a model end stage liver disease (MELD) score, a Child-Pugh score, or a Conn score.

A MELD score uses the patient's values for serum bilirubin, serum creatinine, and the international normalized ratio for prothrombin time (INR) to provide a weighted numeric value to correlate with predicted survival. A score of 10-19 correlates with a 27% mortality rate within three months, and a score of <10 correlates with a 4% mortality within three months. Methods for determination and analysis of MELD scores are well known in the art.

A Child-Pugh score (sometimes the Child-Turcotte-Pugh score) used to assess the prognosis of chronic liver disease, mainly cirrhosis, is an aggregate score of five clinical measures, billirubin, serum albumin, INR, ascites, and hepatic encephalopathy. Each marker is assigned a value from 1-3, and the total value is used to provide a score categorized as A (5-6 points), B (7-9 points), or C (10-15 points), which can be correlated with one and two year survival rates. Methods for determination and analysis of Child-Pugh scores are well known in the art.

A Conn score is an assessment of mental status rather than clinical values to characterize the status of a subject having or suspected of having hepatic encephalopathy.

The language “a prophylactically effective amount” of a compound refers to an amount of a compound provided herein of formula (I) or otherwise described herein which is effective upon single or multiple dose administration to the subject in preventing or treating a bacterial infection or HE.

The language “therapeutically effective amount” refers to an amount of an agent which is effective, upon single or multiple dose administration to the subject to provide a therapeutic benefit to the subject. In another embodiment, the therapeutic benefit is inhibiting a bacterial infection or prolonging the survival of a subject with such a bacterial infection beyond that expected in the absence of such treatment.

Rifaximin exerts a broad antibacterial activity in the gastrointestinal tract against localized gastrointestinal bacteria that cause infectious diarrhea, including anaerobic strains. It has been reported that rifaximin is characterized by a negligible systemic absorption, due to its chemical and physical characteristics (Descombe J. J. et al. Pharmacokinetic study of rifaximin after oral administration in healthy volunteers. Int J Clin Pharmacol Res, 14 (2), 51-56, (1994)).

The presence of hepatic insufficiency has been found to have an effect on in vivo plasma exposure of rifaximin. Thus, making it a criteria for consideration by a healthcare professional (e.g., physician, physician's assistant, nurse practitioner, pharmacist) when prescribing a dose of rifaximin for treatment of a bowel disorder, such as Travelers' diarrhea or IBS. Hepatic insufficiency leads to a clinically statistically significant increase in rifaximin adsorbed by subjects undergoing treatment. One embodiment, disclosed herein are methods of modulating the therapeutic action of rifaximin by selecting a subject suffering from hepatic insufficiency and further providing prophylaxis or treatment for the hepatic insufficiency.

Accordingly, one embodiment is a method of treating Travelers' Diarrhea (TD) in a subject. In this method rifaximin is administered to a subject suffering from a disease that is treatable by rifaximin. The subject is informed that systemic plasma exposure to rifaximin is increased in subjects suffering from hepatic insufficiency in comparison to subjects not suffering from hepatic insufficiency. This information increases the level of safety of administering the rifaximin to the subject. Examples of diseases treatable by rifaximin include Travelers' Diarrhea and Hepatic Encephalopathy.

As used herein, “informing” or “advising” means referring to or providing, published or oral material. For example, providing an active agent with published material to a user; or presenting information orally, for example, by presentation at a seminar, conference, or other educational presentation, by conversation between a pharmaceutical sales representative and a medical care worker, or by conversation between a medical care worker and a patient; or demonstrating the intended information to a user for the purpose of comprehension. Examples of medical care workers include physicians, nurses and nurse practitioners.

One aspect includes providing information to prescribing physicians and patients receiving rifaximin treatment useful in minimizing safety concerns of rifaximin. In this embodiment, the information describes that systemic plasma exposure to rifaximin is increased in subjects suffering from hepatic insufficiency in comparison to subjects not suffering from hepatic insufficiency. In one embodiment, the information is provided on a label. In another embodiment, the information is provided on an information sheet that is given to the patient when a prescription for rifaximin is filled.

Further, the method may also include the step or steps of distributing prescribable doses of rifaximin to pharmacies and distributing educational materials to the pharmacies including pharmacists with the educational material including information as to what the patient needs to know and what the patients must do in order to both avoid any adverse effects of rifaximin while taking the doses.

In addition, the method may include the step or steps of providing guidelines to the pharmacists for counseling patients with regard to what the patient needs to know and what the patient must do in order to safely take their rifaximin dosages. The method may further include the step of requiring acknowledgment of receipt of the educational materials and guidelines from the pharmacists and further acknowledgement of receipt of the educational materials by the patient from the pharmacists.

Another embodiment is a method of using rifaximin for treating a patient's condition. The embodiment includes providing a patient with rifaximin and informing the patient or a medical care worker that systemic plasma exposure to rifaximin is increased in patients suffering from hepatic insufficiency, and that administration of rifaximin to a patient with hepatic insufficiency can affect plasma concentration, safety, or efficacy of rifaximin.

Yet another embodiment includes a method of treating a subject suffering from an indication treatable by rifaximin. This method includes administering rifaximin to the subject and advising the subject that systemic plasma exposure to rifaximin is increased in subjects suffering from hepatic insufficiency in comparison to subjects not suffering from hepatic insufficiency.

### Methods of Treatment

Provided herein are methods of determining a dose of rifaximin for treating, preventing, or alleviating bowel related disorders, particularly Travelers' diarrhea, in a subject further suffering from hepatic insufficiency, e.g. due to hepatic encephalopathy. Bowel related disorders include one or more of hepatic insufficiency, cirrhosis, polycystic liver disease, irritable bowel syndrome, diarrhea, microbe associated diarrhea, Clostridium difficile associated diarrhea, travelers' diarrhea, small intestinal bacterial overgrowth, Crohn's disease, chronic pancreatitis, pancreatic insufficiency, enteritis, colitis, hepatic encephalopathy (or other disease which leads to increases ammonia levels), or pouchitis.

As used herein, the term “hepatic insufficiency” includes diseases and disorders in which a subject has defective functional activity of the liver. Clinically, subjects having hepatic insufficiency have decreased, e.g., statistically significantly decreased, liver function. Hepatic insufficiency often leads to liver failure. One exemplary disease which manifests hepatic insufficiency is hepatic encephalopathy.

As used herein, the term “hepatic encephalopathy” refers to a reversible neuropsychiatric abnormality in the setting of chronic or acute liver failure. When a subject has liver impairment, toxic substances that are normally removed by the liver accumulate in the blood, thereby impairing the function of the brain. These toxic substances are often nitrogenous substances, most notably ammonia. Once in brain tissue, the compounds produce alterations of neurotransmission that affect consciousness and behavior. There are 4 progressive stages of impairment associated with HE that are defined by using the West Haven criteria (or Conn score) which range from Stage 0 (lack of detectable changes in personality) to Stage 4 (coma, decerebrate posturing, dilated pupils). Typical symptoms of hepatic encephalopathy can include impaired cognition, a flapping tremor (asterixis), and a decreased level of consciousness including coma (e.g., hepatic coma), cerebral edema, and, possibly, death. Hepatic encephalopathy is commonly called hepatic coma or portal-systemic encephalopathy in the literature.

As used herein, the term “Travelers' diarrhea” refers to gastrointestinal illness common amongst travelers. The majority of cases are caused by bacterial, viral or protozoan infection. The primary source of infection is ingestion of fecally contaminated food or water. The length of treatment for a particular bowel disorder will depend in part on the disorder. For example, HE may be treated every day for the remainder of a subject's life, travelers' diarrhea may only require treatment duration of 12 to about 72 hours, while Crohn's disease may require treatment durations from about 2 days to 3 months. Dosages of rifaximin will also vary depending on the diseases state.

The identification of those subjects who are in need of prophylactic treatment for bowel disorder is well within the ability and knowledge of one skilled in the art. Certain of the methods for identification of subjects which are at risk of developing a bowel disorder which can be treated by the subject method are appreciated in the medical arts, such as family history, travel history and expected travel plans, the presence of risk factors associated with the development of that disease state in the subject. A clinician skilled in the art can readily identify such candidate subjects, by the use of, for example, clinical tests, physical examination and medical/family/travel history.

A method of assessing the amount of hepatic insufficiency in a subject can include the use of any of the scoring systems provided above, such as a MELD score, a Child-Pugh score, or a Conn score.

In yet another aspect, a method of treating a subject suffering from or susceptible to a bowel disorder comprises administering to a subject in need thereof a therapeutically effective amount of a rifaximin to thereby treat the subject. Upon identification of a subject suffering from or susceptible to a bowel disorder, rifaximin is administered. Rifaximin may be administered, for example, after diagnosis, after an HE event, during an HE event, after diagnosis of minimal HE, or when the critical flicker frequency reaches a level indicative of an HE event, etc. As discussed herein, a physician may choose to alter the dosage of rifaximin administered to a subject for the treatment of a bowel disorder if the subject also has hepatic insufficiency.

Efficacy of a treatment may be measured, for example, as reduction of bacterial overgrowth. Efficacy may also be measured in terms of a reduction of symptoms associated with the bowel disorder, a stabilization of symptoms, or a cessation of symptoms associated with a bowel disorder, for example, a reduction in one or more of nausea, bloating, diarrhea, the severity of the next HE event, and the like, or an increase in one or more of time to next HE event, cognitive ability and the like.

As used herein, the term “terminal elimination rate constant” refers to the first order rate constant describing rifaximin elimination from the body of a subject. This is an overall elimination rate constant describing removal of rifaximin by all elimination processes including excretion and metabolism.

As used herein, the term “plasma concentration” refers to concentration of rifaximin in the plasma of a subject. Plasma concentrations of rifaximin can be determined, for example, using a reversed-phase high performance liquid chromatographic method with tandem quadrupole mass spectrometric detection (LC/MS/MS) as set forth in the Examples. The maximum plasma concentration at steady-state is referred to herein as Cmax and the minimum plasma concentration is referred to as Cmin.

As used herein, the term “clearance rate” refers to the volume of biological fluid completely cleared of drug metabolites as measured in unit time. Elimination occurs as a result of metabolic processes in the kidney, liver, saliva, sweat, intestine, heart, brain, and other locations.

As used herein, alanine aminotransferase also referred to as ALT, refers to a test preformed in order to identify liver damage or liver failure. The level of the enzyme, ALT, is measured to determine if liver damage or disease is present in an individual. Low levels of ALT are normally found in the blood. But when the liver is damaged or diseased, ALT is released into the bloodstream. ALT levels can be measured by methods known to those of skill in the art.

### Pharmaceutical Preparations

Embodiments also provide pharmaceutical compositions, comprising an effective amount of a rifaximin described herein and a pharmaceutically acceptable carrier. In a further embodiment, the effective amount is effective to treat a bacterial infection, e.g., small intestinal bacterial overgrowth, Crohn's disease, hepatic encephalopathy, antibiotic associated colitis, and/or diverticular disease in a subject further suffering from hepatic insufficiency.

For examples of the use of rifaximin to treat Travelers' diarrhea, see Infante R M, Ericsson C D, Zhi-Dong J, Ke S, Steffen R, Riopel L, Sack D A, DuPont, H L. Enteroaggregative Escherichia coli Diarrhea in Travelers: Response to Rifaximin Therapy. Clinical Gastroenterology and Hepatology. 2004;2:135-138; and Steffen R. M.D., Sack D A, M.D., Riopel L, Ph.D., Zhi-Dong J, Ph.D., Sturchler M, M.D., Ericsson C D, M.D., Lowe B, M.Phil., Waiyaki P, Ph.D., White M, Ph.D., DuPont H L, M.D. Therapy of Travelers' Diarrhea With Rifaximin on Various Continents. The American Journal of Gastroenterology. May 2003, Volume 98, Number 5, all of which are incorporated herein by reference in their entirety.

Embodiments also provide pharmaceutical compositions comprising rifaximin and a pharmaceutically acceptable carrier. Doses may be selected, for example on the basis of desired amounts of systemic absorption, elimination rate, plasma concentration and the like. Embodiments of the pharmaceutical composition further comprise excipients, for example, one or more of a diluting agent, binding agent, lubricating agent, disintegrating agent, coloring agent, flavoring agent or sweetening agent. One composition may be formulated for selected coated and uncoated tablets, hard and soft gelatin capsules, sugar-coated pills, lozenges, wafer sheets, pellets and powders in sealed packet. For example, compositions may be formulated for topical use, for example, ointments, pomades, creams, gels and lotions.

In an embodiment, rifaximin is administered to the subject using a pharmaceutically-acceptable formulation, e.g., a pharmaceutically-acceptable formulation that provides sustained delivery of the rifaximin to a subject for at least 12 hours, 24 hours, 36 hours, 48 hours, one week, two weeks, three weeks, or four weeks after the pharmaceutically-acceptable formulation is administered to the subject.

In certain embodiments, these pharmaceutical compositions are suitable for topical or oral administration to a subject. In other embodiments, as described in detail below, the pharmaceutical compositions provided herein may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “pharmaceutically acceptable” refers to rifaximin compositions containing rifaximin and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” includes pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier is preferably “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In solid dosage forms of rifaximin for oral administration (capsules, tablets, pills, dragees, powders, granules and the like), the active ingredient is typically mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such as talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient moistened with an inert liquid diluent.

The tablets, and other solid dosage forms of the pharmaceutical compositions described herein, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of rifaximin include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

In addition to inert diluents, the oral compositions can include adjuvants such as wetting agents, emulsifying and suspending agents, sweetening, flavoring, coloring, perfuming and preservative agents.

Suspensions, in addition to rifaximin may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions for rectal or vaginal administration may be presented as a suppository, which may be prepared by mixing rifaximin with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Compositions which are suitable for vaginal administration also include pessaries, tampons, creams, gels, pastes, foams or spray formulations containing such carriers as are known in the art to be appropriate.

Dosage forms for the topical or transdermal administration of rifaximin includes powders, sprays, ointments, pastes, creams, lotions, gels, solutions, patches and inhalants. Rifaximin may be mixed under sterile conditions with a pharmaceutically-acceptable carrier, and with any preservatives, buffers, or propellants which may be required.

Ointments, pastes, creams and gels may contain, in addition to rifaximin, excipients, such as animal and vegetable fats, oils, waxes, paraffins, starch, tragacanth, cellulose derivatives, polyethylene glycols, silicones, bentonites, silicic acid, talc and zinc oxide, or mixtures thereof

Powders and sprays can contain, in addition to rifaximin, excipients such as lactose, talc, silicic acid, aluminium hydroxide, calcium silicates and polyamide powder, or mixtures of these substances. Sprays can additionally contain customary propellants, such as chlorofluorohydrocarbons and volatile unsubstituted hydrocarbons, such as butane and propane.

Rifaximin can be alternatively administered by aerosol. This is accomplished by preparing an aqueous aerosol, liposomal preparation or solid particles containing the compound. A non-aqueous (e.g., fluorocarbon propellant) suspension could be used. Sonic nebulizers are preferred because they minimize exposing the agent to shear, which can result in degradation of the compound.

Injectable depot forms are made by forming microencapsule matrices of rifaximin in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the rifaximin is administered as a pharmaceutical, to humans and animals, it can be given per se or as a pharmaceutical composition containing, for example, 0. 1 to 99.5% (more preferably, 0.5 to 90%) of active ingredient in combination with a pharmaceutically-acceptable carrier.

Regardless of the route of administration selected rifaximin which may be used in a pharmaceutical compositions provided herein, is formulated into pharmaceutically-acceptable dosage forms by methods known to those of skill in the art.

Actual dosage levels and time course of administration of the active ingredients in the pharmaceutical compositions may be varied so as to obtain an amount of the active ingredient which is effective to achieve the desired therapeutic response for a particular subject, composition, and mode of administration, without being toxic to the subject. An exemplary dose range is from 25 to 3000 mg per day.

A preferred dose of rifaximin is the maximum that a subject can tolerate without developing serious side effects. Preferably, rifaximin is administered at a concentration of about 1 mg to about 200 mg per kilogram of body weight, about 10-about 100 mg/kg or about 40 mg-about 80 mg/kg of body weight. Ranges intermediate to the above-recited values are also intended to be part of the invention.

In exemplary embodiments, subjects are administered rifaximin 1, 2, 3, or 4 times a day. Exemplary dosages include oral dosages of 100, 200, 300, 400, 500, 550, 600, 700, 800, 900, 1000, 1100, 1200, 1300, 1400, 1500, 1600, 1700, 1800, 1900 or 2000 mg of rifaximin. Ranges intermediate to the above-recited values are also intended to be part of the invention. In a specific exemplary embodiment, subjects are administered 600 mg or rifaximin per day. In a further specific embodiment, subjects are administered three 200 mg tablets per day.

In combination therapy treatment, rifaximin and the other drug agent(s) are administered to mammals (e.g., humans, male or female) by conventional methods. The agents may be administered in a single dosage form or in separate dosage forms. Effective amounts of the other therapeutic agents are well known to those skilled in the art. However, it is well within the skilled artisan's purview to determine the other therapeutic agent's optimal effective-amount range. In one embodiment in which another therapeutic agent is administered to an animal, the effective amount of the rifaximin is less than its effective amount in case the other therapeutic agent is not administered. In another embodiment, the effective amount of the conventional agent is less than its effective amount in case the rifaximin is not administered. In this way, undesired side effects associated with high doses of either agent may be minimized. Other potential advantages (including without limitation improved dosing regimens and/or reduced drug cost) will be apparent to those skilled in the art.

In various embodiments, the therapies (e.g., prophylactic or therapeutic agents) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In preferred embodiments, two or more therapies are administered within the same subject's visit.

In certain embodiments, one or more compounds and one or more other therapies (e.g., prophylactic or therapeutic agents) are cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time, optionally, followed by the administration of a third therapy (e.g., prophylactic or therapeutic agent) for a period of time and so forth, and repeating this sequential administration, e.g., the cycle in order to reduce the development of resistance to one of the therapies, to avoid or reduce the side effects of one of the therapies, and/or to improve the efficacy of the therapies.

In certain embodiments, the administration of the same compounds may be repeated and the administrations may be separated by at least 1 day, 2 days, 3 days, 5 days, 10 days, 15 days, 30 days, 45 days, 2 months, 75 days, 3 months, or at least 6 months. In other embodiments, the administration of the same therapy (e.g., prophylactic or therapeutic agent) other than rifaximin may be repeated and the administration may be separated by at least 1 day, 2 days, 3 days, 5 days, 10 days, 15 days, 30 days, 45 days, 2 months, 75 days, 3 months, or at least 6 months.

Certain indications may require longer treatment times. For example, travelers' diarrhea treatment may only last from between about 12 hours to about 72 hours, while a treatment for Crohn's disease may be from between about 1 day to about 3 months. A treatment for hepatic encephalopathy may be, for example, for the remainder of the subject's life span. A treatment for IBS may be intermittent for weeks or months at a time or for the remainder of the subject's life.

### Article of Manufacture

Another embodiment includes articles of manufacture that comprise, for example, a container holding a pharmaceutical composition suitable for oral or topical administration of rifaximin in combination with printed labeling instructions providing a discussion of when a particular dosage form should be administered with food and when it should be taken on an empty stomach. The dosage can be modified for administration to a subject suffering from hepatic insufficiency, or include labeling for administration to a subject suffering from hepatic insufficiency. Exemplary dosage forms and administration protocols are described infra. The composition will be contained in any suitable container capable of holding and dispensing the dosage form and which will not significantly interact with the composition and will further be in physical relation with the appropriate labeling. The labeling instructions will be consistent with the methods of treatment as described hereinbefore. The labeling may be associated with the container by any means that maintain a physical proximity of the two, by way of non-limiting example, they may both be contained in a packaging material such as a box or plastic shrink wrap or may be associated with the instructions being bonded to the container such as with glue that does not obscure the labeling instructions or other bonding or holding means.

Another aspect is an article of manufacture that comprises a container containing a pharmaceutical composition comprising rifaximin wherein the container holds preferably rifaximin composition in unit dosage form and is associated with printed labeling instructions advising of the differing absorption when the pharmaceutical composition is taken with and without food.

In another aspect, provided herein are a article of manufacture or a kit comprising rifaximin, or a pharmaceutical composition thereof, packaged with instructions for administering to subjects having hepatic insufficiency. In one embodiment, the instructions will inform the prescribing physician, a pharmacist, or a subject that subjects having hepatic encephalopathy absorb a larger amount of rifaximin systemically than subject with normal, e.g., fully functional, hepatic systems. In one embodiment, the instructions will advise the prescribing physician that they should determine if the subject has hepatic insufficiency before prescribing rifaximin to a subject to treat a bowel disease. In one embodiment, the instructions will advise the prescribing physician that they should consider altering the dosage or dosing regimen when prescribing rifaximin to a subject having hepatic encephalopathy. Additionally, the instructions may provide prescribing information for subjects with hepatic insufficiency. In another embodiment, the instructions will inform the subject and/or the healthcare provider that there is a difference in the plasma exposure to rifaximin between HE subjects and subjects with normal liver function.

Packaged compositions are also provided, and may comprise a therapeutically effective amount of rifaximin. Rifaximin and a pharmaceutically acceptable carrier or diluent, wherein the composition is formulated for treating a subject suffering from or susceptible to a bowel disorder, and packaged with instructions to treat a subject suffering from or susceptible to a bowel disorder.

Kits are also provided herein, for example, kits for treating a bowel disorder in a subject. The kits may contain, for example, rifaximin and instructions for use when treating a subject for a bowel disorder who is also suffering from hepatic insufficiency. The instructions for use may contain prescribing information, dosage information, storage information, and the like.

Packaged compositions are also provided, and may comprise a therapeutically effective amount of rifaximin and a pharmaceutically acceptable carrier or diluent, wherein the composition is formulated for treating a subject suffering from or susceptible to a bowel disorder, and packaged with instructions to treat a subject suffering from or susceptible to a bowel disorder.

### EXAMPLES

Embodiments of the invention are based, in part, on the demonstration that rifaximin is differentially absorbed in subjects having other clinical conditions, e.g., hepatic encephalopathy. It should be appreciated that the invention should not be construed to be limited to the examples that are now described; rather, the invention should be construed to include any and all applications provided herein and all equivalent variations within the skill of the ordinary artisan.

### Example 1

### Clinical Study of Rifaximin Administration to Subjects with Impaired Liver Function

To determine the effect of impaired liver function on rifaximin efficacy, tests were preformed on subjects having hepatic encephalopathy (HE). HE, also known as hepatic coma or portal-systemic encephalopathy, is a serious, rare, complex, potentially reversible, neuropsychiatric syndrome associated with advanced liver disease. Nitrogenous substances, most notably ammonia, gain access to the systemic circulation as a result of decreased hepatic function or portal-systemic shunts. Once in brain tissue, the compounds produce alterations of neurotransmission that affect consciousness and behavior. There are four progressive stages of impairment associated with HE that are defined by using the West Haven criteria (or Conn score) which range from Stage 0 (lack of detectable changes in personality) to Stage 4 (coma, decerebrate posturing, dilated pupils).

Management of patients with chronic HE includes: 1) provision of supportive care, 2) identification and removal of precipitating factors, 3) reduction of nitrogenous load from the gut, and 4) assessment of the need for long term therapy. The nitrogenous load from the gut is typically reduced using nonabsorbable disaccharide (lactulose) and/or antibiotics. Although lactulose is considered a first-line treatment in the United States, it is not currently approved for either the treatment or prevention of HE. Rifaximin is an attractive therapy for the treatment of patients with HE because of its demonstrated effectiveness and because of disadvantages of systemic antibiotics and nonabsorbable disaccharides. Disadvantages of chronic systemic antibiotic therapy include nephrotoxicity and ototoxicity, and disadvantages of lactulose therapy include dehydration due to diarrhea (a precipitating factor of HE), overly sweet taste, and GI side effects.

In this example, rifaximin was dosed in an outpatient setting at 550 mg BID (for a total daily dose of 1100 mg rifaximin). Subjects were dosed with 550 mg of rifaximin BID for at least 7 consecutive days prior to the day of pharmacokinetic sampling.

To ensure steady-state plasma concentrations, blood sampling for pharmacokinetic analyses was performed after at least 7 consecutive days of rifaximin 550 mg BID dosing. Blood samples for pharmacokinetic analyses were collected on a single day at after at least 7 consecutive days of 100% compliance with the rifaximin 550 mg BID dosing regimen. Multiple samples for pharmacokinetic analyses were collected over 12 hours (e.g., predose and at 1, 2, 4, 6, 8, 10 and 12 hours after dosing) to permit steady-state characterization of the plasma rifaximin concentration-time profile. Subjects fasted overnight (no food for approximately 10 hours) prior to administration of rifaximin and were given a standardized light meal 1 hour following administration of study drug (subsequent to the planned 1 hour plasma collection).

Pharmacokinetic parameters of rifaximin in plasma were calculated using noncompartmental methods (e.g., standard model-independent approach).

Pharmacokinetic sample collection occurred on a single day following at least 7 consecutive days of 100% compliance with the rifaximin 550 mg BID dosing regimen. A total of 8 blood samples were collected over 12 hours (e.g., predose and at 1, 2, 4, 6, 8, 10, and 12 hours after dosing) to permit characterization of the individual plasma rifaximin concentration-time profile over the 12-hour dosing interval.

Plasma concentrations of rifaximin were determined using a reversed-phase high performance liquid chromatographic method with tandem quadrupole mass spectrometric detection (LC/MS/MS) using a validated analytical procedure. The lower limit of quantification (LOQ), deviation of calibration standards from the theoretical value, and precision were established using standard methods.

Pharmacokinetic parameters of rifaximin in plasma were calculated using WinNonlin® Enterprise (Version 5.2).

Pharmacokinetic parameters were calculated using noncompartmental methods (e.g., standard model-independent approach). The following steady-state pharmacokinetic parameters for rifaximin in plasma were calculated using actual concentration-time profiles for each subject:

| Parameter | Definition |
|---|---|
| AUC_{τ} | Area under the concentration versus time curve from time 0 |
| | (pre-dose) over the 12 hours dosing interval tau (τ) calculated |
| | using the linear trapezoid rule (also referred as AUC₀₋₁₂). |
| Cₘₐₓ | Maximum plasma concentration at steady-state. Also referred |
| | to as Cmaxₛₛ. |
| Cₘᵢₙ | Minimum plasma concentration at steady-state. Also referred |
| | to as Cminₛₛ. |
| Tₘₐₓ | Time maximum plasma concentration at steady-state. Also |
| | referred to as Tmaxₛₛ. |

Other parameters such as apparent oral clearance (CL/F) and terminal or disposition half-life (t_{1/2}) were estimated if adequate data was available. In addition to the planned analysis, the AUC from time 0 (pre-dose) to the last measurable concentration (AUC₀₋ₜ) was also calculated.

Individual plasma concentration and pharmacokinetic parameters of rifaximin were summarized for the overall pharmacokinetic population and by hepatic impairment severity using Child-Pugh scores (A and B) with descriptive statistics (e.g., N, mean, SD, CV %, median, min, max, Geometric mean).

Demographics and other baseline characteristics were summarized for subjects by hepatic impairment severity using Child-Pugh scores (A and B) and Model End-Stage Liver Disease (MELD) score with descriptive statistics. Baseline characteristics included albumin, alkaline phosphate, alanine aminotransferase (ALT), aspartate aminotransferase (AST), international normalized ratio (INR), serum creatinine, and serum total bilirubin, where baseline was defined as last available assessment prior to the first dose of rifaximin.

Rifaximin pharmacokinetic parameters AUC_{τ} and Cₘₐₓ in subjects with Child-Pugh scores A and B (e.g., mild and moderate liver impairment) were compared using an analysis of variance (ANOVA) model.

A paired ANOVA was used to evaluate concentration values of rifaximin measured at predose and at 12 hours postdose to assess possible differences in steady-state rifaximin concentrations.

A total of 25 subjects were included in the pharmacokinetic evaluable population and evaluated for safety.

Eighteen (18) of 25 subjects (72.0%) had mild hepatic impairment at baseline (e.g., Child-Pugh score A). The remaining 7 subjects (28.0%) had moderate hepatic impairment (e.g., Child-Pugh score B) at baseline.

Rifaximin pharmacokinetic parameters were compared to results from a separate study on healthy subjects with normal hepatic function.

Subject Demographics and Baseline Characteristics

Table 1 summarizes demographics for all enrolled subjects. A total of 25 subjects were enrolled in the study; 17 subjects (68.0%) were male and 8 subjects (32.0%) were female. The mean age among participating subjects was 58 years (range 45 to 68 years). Twenty-two subjects (88.0%) were white, and the remaining 3 subjects (12.0%) were black. Seven of 25 subjects (28.0%) were of hispanic ethnicity.

Eighteen (18) subjects had a Child-Pugh classification of A and 7 subjects had a Child-Pugh classification of B. Fifteen (15) subjects had a baseline MELD score of <11 and 10 subjects had a baseline MELD score between 11 and 18 (inclusive). The majority of subjects participating had a Conn Score of 0 (22/25; 88.0%) at baseline for the pharmacokinetic substudy; 3 of 25 subjects (12.0%) had a Conn Score of 1 at baseline.

TABLE 1
Subject Demographics and Baseline Characteristics -
All Enrolled Subjects

| Characteristic | N = 25 |
|---|---|
| N | 25 |
| Mean(±SD) age, years | 58 (±5.34) |
| Sex: n (%) | |
| Male | 17 (68.0) |
| Female | 8 (32.0) |
| Race: n (%) | |
| White | 22 (88.0) |
| Black or African American | 3 (12.0) |
| Child-Pugh Score: n (%) | |
| A | 18 (72.0) |
| B | 7 (28.0) |
| MELD Score: n (%) | |
| <11 | 15 (60.0) |
| 11-18 | 10 (40.0) |
| Conn Score | |
| Grade 0 | 22 (88.0) |
| Grade 1 | 3 (12.0) |

Abbreviations:
MELD = model end-stage liver disease.

Overall, demographic characteristics were comparable for Child-Pugh A and Child-Pugh B subjects. Baseline demographics were also generally similar for subjects who had a baseline MELD score of <11 and subjects who had a baseline MELD score between 11 and 18 (inclusive). A higher proportion of subjects with a MELD score between 11 and 18 were Hispanic (50.0% vs. 13.3%) compared with subjects with a MELD score ≦10.

Baseline laboratory findings were consistent with impaired liver function among subjects. Results of baseline liver function tests indicated greater hepatic impairment among subjects categorized as Child-Pugh B compared with subjects categorized as Child-Pugh A and greater hepatic impairment among subjects with a MELD score between 11 and 18 compared with subjects with a MELD score <11. Specifically, Child-Pugh B subjects and subjects with a MELD score of 11-18 had noticeably higher baseline values for alkaline phosphatase, AST, and direct and total bilirubin at baseline.

On the day preceding the pharmacokinetic collection, the majority of subjects received their 2 rifaximin doses at an interval of approximately 12 hours apart. The shortest interval between doses for any subject was 10 hours; the longest interval for any subject was 13.55 hours. The 2^{nd} rifaximin dose was administered without regard to the evening meal, either before food (13 subjects) or after food (12 subjects).

On the day of pharmacokinetic sampling, the morning rifaximin dose was administered following at least 10 hours of overnight fasting. All subjects had a light meal served 1 hour postdose, subsequent to the 1 hour pharmacokinetic plasma sampling time point. The next rifaximin dose was taken immediately after the 12-hour pharmacokinetic plasma sampling time point, with 1 exception.

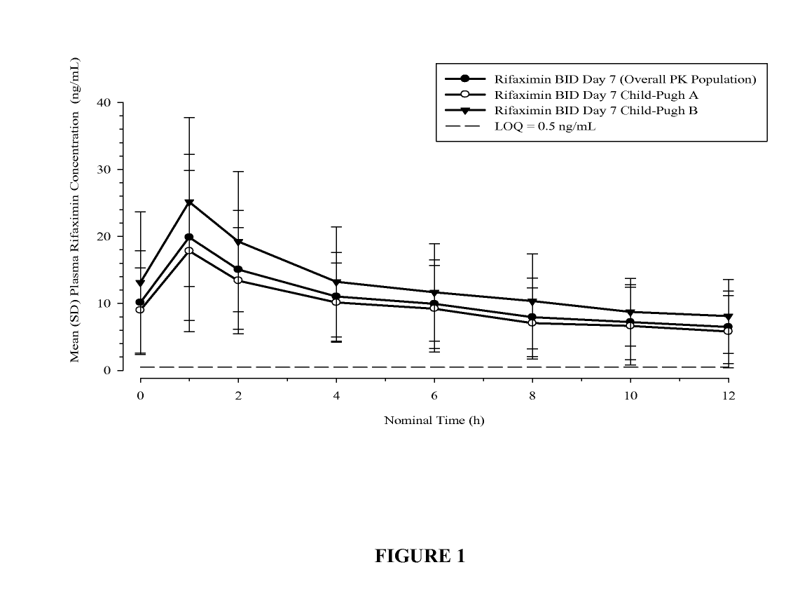

Mean plasma concentrations of rifaximin peaked at 1 hour after drug administration and then declined slowly over 12 hours (FIG. 1). Rifaximin plasma concentrations were above the limit of quantification (LOQ) of the assay over the entire 12-hour sampling interval in all subjects. A total of 5 subjects displayed double peak plasma concentration profiles.

Rifaximin pharmacokinetic parameters at steady-state in subjects with hepatic impairment classifications of Child-Pugh A and Child-Pugh B

Table 2 summarizes pharmacokinetic parameters of rifaximin following at least 7 days of treatments in subjects with impaired liver function by Child-Pugh scores and for the overall pharmacokinetic population. A column including the values determined for the healthy subjects in a separate study is provided to facilitate comparison.

TABLE 2
Mean (±SD) Plasma Pharmacokinetic Parameters of
Rifaximin in Subjects with Liver Impairment

| | Hepatic Insufficient | | | |
|---|---|---|---|---|
| | Child-Pugh A | Child-Pugh B | | |
| | (Mild) | (Moderate) | Overall | Healthy Volunteers |
| Parameters | N = 18 | N = 7 | N = 25 | N = 14 |
| AUC₀₋ₜ (ng · h/mL) | 113 (68.2) | 156 (93.0) | 125 (76.4) | 11.5 (6.44) |
| AUCₜₐᵤ (ng · h/mL) | 118 (67.8)^{a} | 161 (101)^{b} | 130 (77.6)^{c} | 12.3 (4.76) |
| Cₘₐₓ (ng/mL) | 19.5 (11.4) | 25.1 (12.6) | 21.1 (11.8) | 3.41 (1.62) |
| Cₘᵢₙ (ng/mL) | 5.13 (1.04) | 7.90 (5.35) | 5.91 (4.49) | 0.275 (0.333) |
| Tₘₐₓ (h)^{d} | 1.00 (0.933, 10.0) | 1.00 (0.967, 1.00) | 1.00 (0.933, 10.0) | 0.76 (0.50-4.00) |
| t_{1/2} (h)^{e} | 8.12 (3.58)^{f} | 10.5 (1.50)^{g} | 8.64 (3.63)^{h} | 4.17 (3.30)^{h} |
| CL/F (L/min) | 122 (101)^{a} | 70.6 (29.2)^{b} | 109 (90.1)^{c} | 863 (364) |

^{a}n = 17
^{b}n = 6
^{c}n = 23
^{d}Median (Min, Max)
^{e}Harmonic mean (pseudo SD)
^{f}n = 14
^{g}n = 5
^{h}n = 19

### Comparisons between Subjects with Child-Pugh a (Mild Impairment) Versus Child-Pugh B (Moderate Impairment) and between Subjects with MELD Scores of <11 (Mild Impairment) Versus 11 to 18 (Moderate Impairment)

Mean AUC_{τ} and Cₘₐₓ values in subjects with Child-Pugh score B (161 ng*h/mL and 25.1 ng/mL, respectively) were approximately 36% and 29% higher than those observed in subjects with Child-Pugh score A (118 ng*h/mL and 19.5 ng/mL, respectively). The elimination rate of rifaximin in subjects with Child-Pugh B score was approximately 29% longer than that observed in subjects with Child-Pugh A score (10.5 h vs. 8.12 h). The pharmacokinetics of rifaximin were characterized by an inter-subject coefficient of variability (CV %) for AUC_{τ} and Cₘₐₓ ranging from approximately 50 to 60%. This was in agreement with the variability previously observed in healthy subjects e.g., CV % of 45% to 60%. Rifaximin pharmacokinetic parameters AUC_{τ} and Cₘₐₓ in subjects with Child-Pugh scores A and B (mild and moderate hepatic impairment, respectively) were compared using an ANOVA model. For cases where the AUC_{τ} could not be calculated the corresponding AUC₀₋ₜ values were used for inferential statistics.

The results of the one-way ANOVA analysis are summarized in Table 3. The ratio of AUC_{τ} geometric LSM for Child-Pugh Score B to Child-Pugh Score A was 151.2% with 90% confidence intervals of 98.8% to 231.5% (p=0.1092). The ratio of Cₘₐₓ geometric LSM for Child-Pugh Score B to Child-Pugh Score A was 149.9% with 90% confidence intervals of 98.8% to 227.5% (p=0.1096). Confidence intervals for the ratios of LSM were very large given the inter-subject variability in AUC_{τ} and Cₘₐₓ parameters in both populations.

TABLE 3
Effect of Hepatic Impairment Scores (Child-Pugh A versus Child-Pugh B)
on Main Pharmacokinetic Parameters of Rifaximin

| | Geometric LSM | | | | | | |
|---|---|---|---|---|---|---|---|
| | (ng/mL) | | Ratio of | | | | Inter- |
| Pharmacokinetic | Child- | Child- | LSM (B/A) | 90% CI | | Variance | Subject |
| Parameter | Pugh A | Pugh B | (%) | (%) | p value | Assumption | CV (%) |
| AUC_{τ} (ng * h/mL) | 92.44 | 139.80 | 151.2 | (98.8, 231.5) | 0.1092 | Child-Pugh A | 81.8 |
| | | | | | | Child-Pugh B | 49.6 |
| Cₘₐₓ | 15.41 | 23.11 | 149.9 | (98.8, 227.5) | 0.1096 | Child-Pugh A | 91.5 |
| (ng/mL) | | | | | | Child-Pugh B | 43.6 |

Covariate analyses indicated that biochemical markers of impaired hepatic function, e.g., elevated albumin, total bilirubin, and international normalized ratio values correlated with elevated rifaximin systemic exposure (AUCₜₐᵤ and Cₘₐₓ) and decreased oral clearance (CL/F).

The pharmacokinetics of rifaximin were evaluated in subjects with impaired liver function. After receiving the same dosing regimen (e.g., 550 mg BID), rifaximin systemic exposure values (AUCₜₐᵤ) at steady-state in subjects with Child-Pugh A and B were approximately 9.6- and 13.1-fold higher, respectively, than those observed in healthy subjects at steady-state.

Systemic exposure was compared using a different method to assess liver function, MELD score. The ratios of geometric LSMs and 90% CIs for AUC_{τ} and Cₘₐₓ were determined for subjects with MELD score of <11 (n=15) versus MELD score of 11 to 18 (n=10). Results of this analysis (see Table 4) showed that systemic exposure was statistically significantly higher (p<0.05) in subjects with moderate hepatic impairment when compared with mild hepatic impairment when MELD score was used to rate hepatic function. The ratio of AUC_{τ} for MELD score <11 versus 11 to 18 was 168.22% with 90% CIs of 110.5% to 256.2% (p=0.0451); and the ratio of Cₘₐₓ ratio was 178.12% with 90% CIs of 116.7% to 271.8% (p=0.0283). The correlation between MELD score and Child-Pugh category in the 25 subjects who participated in the substudy was mild (Correlation Coefficient: p=0.399).

TABLE 4
Effect of Hepatic Impairment Scores (MELD score <11 versus 11 to 18)
on Main Pharmacokinetic Parameters of Rifaximin

| | Geometric LSM | | | | |
|---|---|---|---|---|---|
| | (ng/mL) | | Ratio of | | |
| Pharmacokinetic | | MELD 11 to | LSM (11-18/<11) | 90% CI | |
| Parameter | MELD <11 | 18 | (%) | (%) | p value |
| AUC_{τ} (ng * h/mL) | 84.30 | 141.81 | 168.22 | (110.5, 256.2) | 0.0451 |
| Cₘₐₓ (ng/mL) | 13.70 | 24.41 | 178.12 | (116.7, 271.8) | 0.0283 |

Rifaximin was rapidly absorbed, with peak plasma concentration observed at 1 hour post-dose in the vast majority of subjects. A total of 3 subjects in the Child-Pugh A group had delayed rifaximin absorption, with peak plasma concentration observed between 6 and 10 hours post dose.

Comparisons to Subjects with Normal Hepatic Function

Results from the current study were compared with historical data from subjects with normal hepatic function. Arithmetic mean (±SD) pharmacokinetic parameters of rifaximin 550 mg multiple-dose BID in healthy subjects are presented in Table 5.

Rifaximin exposure values (AUC_{τ}) in subjects with Child-Pugh score A and B (118 and 161 ng*h/mL, respectively) were approximately 9.6- and 13.1-fold higher than that observed in healthy subjects following twice daily oral doses of 550 mg (12.3 ng*h/mL), respectively. Except for t_{1/2}, intersubject variabilites in the pharmacokinetics of healthy subjects were generally similar to those measured in subjects with hepatic impairment.

TABLE 5
Arithmetic Mean (± SD) Pharmacokinetic Parameters of Rifaximin
550 mg Multiple-Dose BID in Healthy Subjects

| | Healthy Volunteers |
|---|---|
| Parameters | N = 14 |
| AUC₀₋ₜ (ng * h/mL) | 11.5 (6.44) |
| AUC_{τ} (ng * h/mL) | 12.3 (4.76) |
| Cₘₐₓ (ng/mL) | 3.41 (1.62) |
| Cₘᵢₙ (ng/mL) | 0.275 (0.333) |
| Tₘₐₓ (h)^{a} | 0.76 (0.50-4.00) |
| t_{1/2} (h)^{b} | 4.17 (3.30) |
| CL/F (L/min) | 863 (364) |

^{a}Median (Min, Max),
^{b}Harmonic mean (pseudo SD)

### Comparison of Predose Concentrations to 12 Hours Postdose on the Day of the Pharamacokinetic Substudy

Results of the paired ANOVA for the assessment of predose concentrations at 0 and 12 hours are presented in Table 6.

These results indicate that the 12-hour post-dose concentration values of rifaximin were reduced by 37.8% as compared to the morning pre-dose concentration (p<0.0001). Co-administration with a meal was reported to increase rifaximin extent of absorption by approximately 2-3-fold. The morning dose was administered under fasting conditions.

TABLE 6
Paired Analysis of Variance (ANOVA) Evaluation of ln-Transformed
Concentrations of Rifaximin at Predose and at 12 Hours Post-Dose

| Time | Geometric | Ratio of LSM (%) | 90% CI | | Inter-Subject |
|---|---|---|---|---|---|
| of Sample (h) | LSM (ng/mL) | (12 h/0 h) | (%) | p value | CV (%) |
| 0 | 7.72 | 62.2 | (52.0, 74.4) | 0.0001 | 38.4 |
| 12 | 4.80 | | | | |

### Covariate Analyses

A multivariate linear regression model was developed to evaluate the effect of various covariates on the rifaximin AUC_{τ}, Cₘₐₓ, and CL/F. The following covariates were tested in the model: Child-Pugh score and laboratory test results (albumin, alkaline phosphatase, ALT, AST, creatinine clearance, serum creatinine, INR, and total bilirubin). The covariates chosen for the analysis are known indicators of hepatic and renal function. A visual diagnostic was performed to detect potential trends between covariates of interest and AUC_{τ}, Cₘₐₓ, and CL/F.

Results are presented in Tables 7-9 below.

The covariate analyses indicated that biochemical markers of impaired hepatic function, e.g., elevated albumin, total bilirubin, and INR values correlated with elevated rifaximin systemic exposure (AUC_{τ} and Cₘₐₓ) and decreased oral clearance (CL/F) in this study.

The model with the highest R² included albumin, total bilirubin, INR, and ALT (R²=53.6%, Cp=4.4101).

Based on the analyses of the models, it was decided that the parsimonious model would include only albumin, total bilirubin, and INR. The final model for AUC_{τ} is presented in Table 7.

TABLE 7
Relationship Between AUC_{τ} of Rifaximin and Covariates -
Parsimonious Model

| | Final Multivariate Model | | | |
|---|---|---|---|---|
| | | | | Standard- |
| Effect | Estimate | 95% CI | p value | Error |
| Intercept | 8.4175 | (5.0768; 11.7582) | <0.0001 | 1.6064 |
| Albumin | −0.0573 | (−0.1130; −0.0017) | 0.0440 | 0.0268 |
| Total Bilirubin | 0.0173 | (−0.0035; 0.0381) | 0.0988 | 0.0100 |
| INR | −1.7432 | (−3.1909; −0.2956) | 0.0206 | 0.6961 |

The model with 3 parameters having the highest R² included total bilirubin, INR, and ALT (R²=39.1%, Cp=3.7193). Within the subset of models with 4 parameters; the model with the highest R² included albumin, total bilirubin, INR, and ALT (R²=46.9%, Cp=3.0598). Given that the R² was higher for the model with 4 parameters; it was decided that the parsimonious model would include 4 parameters: albumin, total bilirubin, INR, and ALT. The final model is presented in Table 8.

TABLE 8
Relationship Between Cₘₐₓ of Rifaximin and Covariates -
Parsimonious Model

| | Final Multivariate Model | | | |
|---|---|---|---|---|
| Effect | Estimate | 95% CI | p value | Standard-Error |
| Intercept | 6.5350 | (2.7734; 10.2966) | 0.0017 | 1.8033 |
| Albumin | −0.0515 | (−0.1140; 0.0111) | 0.1016 | 0.0300 |
| Total | 0.0207 | (−0.0022; 0.0435) | 0.0742 | 0.0110 |
| Bilirubin | | | | |
| INR | −2.0654 | (−3.7205; −0.4104) | 0.0170 | 0.7934 |
| ALT | 0.0031 | (−0.0004; −0.0066) | 0.0819 | 0.0017 |

Within the subset of models with 4 parameters; the model with the highest R² included albumin, total bilirubin, INR, and ALT (R²=54.9%, Cp=3.4103). Results of this model are presented in Table 9.

TABLE 9
Relationship Between CL/F of Rifaximin and Covariates -
Parsimonious Model

| | Final Multivariate Model | | | |
|---|---|---|---|---|
| Effect | Estimate | 95% CI | p value | Standard-Error |
| Intercept | −0.8934 | (−4.6780; 2.8911) | 0.6259 | 1.8014 |
| Albumin | 0.0783 | (0.0185; 0.1381) | 0.0131 | 0.0285 |
| Total | −0.0185 | (−0.0390; 0.0019) | 0.0732 | 0.0097 |
| Bilirubin | | | | |
| INR | 2.5949 | (0.8604; 4.3295) | 0.0056 | 0.8256 |
| ALT | −0.0028 | (−0.0060; 0.0005) | 0.0925 | 0.0016 |

### Example 2

### A Randomized, Double-Blind, Dose Finding Study to Evaluate the Efficacy, Tolerability and Safety of Rifaximin in Patients with Grade I, II or III Hepatic Encephalopathy

A pharmacokinetic investigation was performed in subjects with HE in a dose-finding study. A total of 54 subjects (32 male, 22 female, age 32 through 82 years) were included in the study and received 200, 400, or 800 mg rifaximin TID (200 mg tablets) corresponding to daily doses of 600, 1200, and 2400 mg, respectively, for 7 consecutive days. Rifaximin plasma and urine concentrations were measured by LC-MS/MS (LLOQ=0.5 ng/mL).

The urine recovery of rifaximin is provided in Table 10.

TABLE 10
Urinary Recovery of Rifaximin During the 24-Hour Collection
Interval After Last Dose

| | Number | Mean Drug | |
|---|---|---|---|
| | of | Recovery | Mean (Range) |
| Dosage | Subjects | (mg) | % Recovery |
| 200 mg TID × 7 days | 18 | 0.37 | 0.061% (0.003-0.229%) |
| 400 mg TID × 7 days | 19 | 1.20 | 0.100% (0.002-0.295%) |
| 800 mg TID × 7 days | 17 | 1.35 | 0.056% (0.002-0.320%) |

There was no relationship between the administered dose and the amount of rifaximin recovered in urine. In the 24-h urine collected after the last (third) 200, 400, and 800 mg dose on the last administration day, Day 7, the mean (SD) amount of rifaximin recovered in the urine ranged from 0.06% (±0.66%) through 0.1% (±0.093%) of dose and these values are consistent with the rifaximin recovered (e.g., 0.030%±0.020% dose) after a single 400 mg radiolabeled dose.

Mean maximum rifaximin plasma concentrations of 2.7, 10.5, and 13.5 ng/mL were measured 3 h after the first single dose of 200, 400, and 800 mg rifaximin, respectively.

### Example 3

### Rifaximin Absorption

A study was performed in hepatically impaired subjects. Mean AUCₜₐᵤ and Cₘₐₓ values in subjects with Child-Pugh score B (161 ng·h/mL and 25.1 ng/mL, respectively) were approximately 36% and 29% higher than those observed in subjects with Child-Pugh score A (118 ng·h/mL and 19.5 ng/mL, respectively). The elimination t_{1/2} of rifaximin in subjects with Child-Pugh B score was approximately 29% longer than that observed in subjects with Child-Pugh A score (10.5 h vs. 8.12 h). Rifaximin pharmacokinetic parameters had inter-subject coefficient of variability percentages (CV %) for AUC₀₋ₜₐᵤ and Cₘₐₓ ranging from approximately 50% to 60% in both subpopulations. This was in agreement with the variability previously observed in healthy subjects, e.g., CV % of 45% through 60%.

Rifaximin was rapidly absorbed, with peak plasma concentration observed at 1 h post-dose in the vast majority of subjects. A total of 3 subjects in the Child-Pugh A group had delayed rifaximin absorption, with peak plasma concentration observed between 6 and 10 h post-dose. Several subjects displayed flat or double-peak plasma concentration profiles of rifaximin. Abnormalities of gastrointestinal motility and of bile secretion in subjects with cirrhosis and HE may potentially explain delayed/prolonged rifaximin absorption observed in this study.

Results of the multiple linear regression models showed that biochemical markers of hepatic function, e.g., elevated albumin, total bilirubin, and International Normalized Ratio, correlated with increased rifaximin systemic exposure (AUCₜₐᵤ and Cₘₐₓ) and decreased oral clearance (CL/F). A positive correlation between baseline alanine aminotransferase and Cₘₐₓ was also observed.

In a separate study, the pharmacokinetic parameters were studied. This population included 18 subjects (72%) with mild hepatic impairment (Child-Pugh A) and 7 subjects with moderate hepatic impairment (Child-Pugh B). The healthy subject study included 28 subjects.

Rifaximin exposure values (AUCₜₐᵤ) in subjects with Child-Pugh score A and B (118 and 161 ng·h/mL, respectively) were approximately 9.6- and 13.1-fold higher, respectively, than those observed in healthy subjects following twice daily oral doses of 550 mg (12.3 ng·h/mL). Except for t_{1/2}, intersubject variability in the pharmacokinetics of healthy subjects were generally similar to those measured in subjects with hepatic impairment.

### Incorporation by Reference

The contents of all references, patents, pending patent applications and published patents, cited throughout this application are hereby expressly incorporated by reference.

### Equivalents

Those skilled in the art will recognize, or be able to ascertain using no more than routine experimentation, many equivalents to the specific embodiments provided herein described herein. Such equivalents are intended to be encompassed by the following claims.

1. A method of treating Travelers' Diarrhea (TD) in a subject, comprising:
administering rifaximin to a subject suffering from Travelers' Diarrhea; and
informing the subject that systemic plasma exposure to rifaximin is increased in subjects suffering from hepatic insufficiency in comparison to subjects not suffering from hepatic insufficiency.

2. The method of claim 1, wherein the increase in systemic plasma exposure to rifaximin comprises a decrease in the terminal elimination rate constant of rifaximin.

3. The method of claim 1, wherein the increase in systemic plasma exposure to rifaximin comprises an increase in plasma concentration of rifaximin.

4. The method of claim 1, wherein the increase in systemic plasma exposure to rifaximin comprises a decrease in the clearance rate of rifaximin.

5. The method of claim 1, wherein administering rifaximin to the subject comprises administering 600 mg of rifaximin per day to the subject.

6. The method of claim 5, wherein administering 600 mg of rifaximin per day to the subject comprises administering three 200 mg tablets to the subject.

7. The method of claim 1, wherein informing the subject comprises providing oral instructions that systemic plasma exposure to rifaximin is increased in subjects suffering from hepatic insufficiency.

8. The method of claim 1, wherein informing the subject comprises providing written instructions that systemic plasma exposure to rifaximin is increased in subjects suffering from hepatic insufficiency.

9. A method of using rifaximin for treating a patient having Traveler's Diarrhea, comprising providing a patient with rifaximin; and informing the patient or a medical care worker that systemic plasma exposure to rifaximin is increased in patients suffering from hepatic insufficiency, and that administration of rifaximin to a patient with hepatic insufficiency can affect plasma concentration, safety, or efficacy of rifaximin.

10. The method of claim 9, wherein the patient is provided with 200 mg tablets of rifaximin.

11. The method of claim 9, wherein the method comprises informing patients with model end stage liver disease that administration of rifaximin can affect plasma concentration, safety, or efficacy of rifaximin.

12. The method of claim 9, wherein informing the patient or the medical care worker comprises informing the patient or medical worker that rifaximin plasma concentrations (Cₘₐₓ) increase in patients with hepatic insufficiency.

13. The method of claim 9, wherein informing the patient or the medical care worker comprises informing the patient or medical worker that terminal elimination rate constant of rifaximin decreases in patients with hepatic insufficiency.

14. The method of claim 9, wherein patients suffering from hepatic insufficiency are patients with an elevated level of alanine aminotransferase (ALT).

15. The method of claim 9, wherein patients suffering from hepatic insufficiency are patients with a Child-Pugh A score.

16. The method of claim 9, wherein patients suffering from hepatic insufficiency are patients with a Child-Pugh B score.

17. (canceled)

18. (canceled)

19. A method of treating a subject suffering from Traveler's Diarrhea, comprising administering rifaximin to the subject and advising the subject that systemic plasma exposure to rifaximin is increased in subjects suffering from hepatic insufficiency in comparison to subjects not suffering from hepatic insufficiency.

20. (canceled)
